(19) United States
(12) Patent Application Publication
(10) Pub. No.: US 2025/0241425 A1
(43) Pub. Date: Jul. 31, 2025

(54) COSMETIC PRODUCT COMPRISING A FLEXIBLE CONTAINER AND AN AQUEOUS OXIDATIVE COMPOSITION CONTAINED THEREIN

(71) Applicant: KAO CORPORATION, Tokyo (JP)

(72) Inventors: Diana BAUER, Darmstadt (DE); Peter BAUER, Darmstadt (DE); Jens HEILMANN, Darmstadt (DE)

(73) Assignee: KAO CORPORATION, Tokyo (JP)

(21) Appl. No.: 18/833,758

(22) PCT Filed: Jan. 26, 2023
(86) PCT No.: PCT/EP2023/051889

§ 371 Date: Jul. 26, 2024

(30) Foreign Application Priority Data
Jan. 27, 2022 (EP) 22153653.5

(51) Int. Cl. A45D 34/00; A61K 8/22; A61K 8/49; A61Q 5/08; A61Q 5/10; B65B 3/04; B65B 7/28; B65D 75/00; B65D 75/56; B65D 75/58

(52) U.S. Cl. CPC A45D 34/00; A61K 8/22; A61K 8/4953; A61Q 5/08; A61Q 5/10; B65B 3/04; B65B 7/28; B65D 75/008; B65D 75/566; B65D 75/5883; A61K 2800/87; A61K 2800/882; B65D 2575/565; B65D 2575/586

(57) ABSTRACT

A cosmetic product comprising a flexible container and an aqueous oxidizing composition A having a pH in the range of 1 to 6 contained in the flexible container, the aqueous oxidizing composition includes at least one oxidizing agent and at least one compound according to Formula (I):

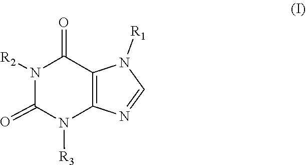

where R₁, R₂, and R₃ are each independently selected from H and CH₃.

### FIELD OF THE INVENTION

The present invention relates to a cosmetic product comprising a flexible container and an aqueous oxidizing composition having a pH in the range of 1 to 6 contained in the flexible container, the composition comprising a) one or more oxidizing agent(s), preferably hydrogen peroxide and b) one or more xanthine compound(s).

### BACKGROUND OF THE INVENTION

A decisive factor to combat climate change is the reduction of fossil fuel consumption, thereby reducing greenhouse gas emissions. The consumer goods industry is, therefore, implementing strategies to enhance product sustainability to offer solutions for reducing its own and the customer's carbon footprint.

One approach is the reduction of packaging material weight leading to a lower total product weight. A lower material consumption saves natural resources during manufacturing, but also reduces emissions in the logistics chains.

However, not all product types are suitable for filling into reduced weight packaging or natural material-based packaging. For products with high chemical activity a safety margin is needed to prevent premature leakage and breaks caused by plastic polymer degradation.

In WO2021/084083, WO2021/084085, and EP3815673 applicant has disclosed aqueous oxidizing compositions comprising xanthine compounds.

Nevertheless, there still is a real need to develop products having a reduced carbon footprint, while allowing for a safe packaging.

### SUMMARY OF THE INVENTION

The first object of the present invention is a cosmetic product comprising a flexible container and an aqueous oxidizing composition A having a pH in the range of 1 to 6 contained in the flexible container, the composition comprising:
a) one or more oxidizing agent(s), preferably hydrogen peroxide,
b) one or more compound(s) according to the following structure:

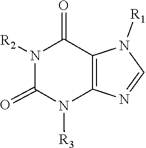

wherein R₁, R₂, and R₃ are independently selected from H and CH₃, and/or their mixtures.

The second object of the present invention is a process of making a cosmetic product having a flexible container filled with an aqueous oxidizing composition A comprising the steps of:
(i) forming a closed flexible container as defined in any above from a film material and fixing the dispensing means to or between the walls of the flexible container,
(ii) filling the flexible container with the aqueous oxidizing composition A as defined above by injecting the liquid composition into the flexible container through the dispensing means,
(iii) sealing the flexible container by closing the dispensing means, preferably with a detachable cap.

The third object of the present invention is a kit-of-parts comprising the cosmetic product as defined above and one more composition selected from:
an aqueous composition B having a pH in the range of 7 to 12 and comprising one or more oxidative dye precursors and/or oxidative dye couplers,
a bleach powder composition C comprising one or more persalt(s) and/or peroxy salt(s) and one or more alkalizing agent(s),
an aqueous lightening composition D comprising one or more alkalizing agent(s) and having a pH in the range of 7 to 12,
an aqueous composition E having a pH in the range of 3 to 12 comprising one or more reducing agent(s) and one or more alkalizing agent(s).

### BRIEF DESCRIPTION OF THE FIGURES

FIG. 1: depicts a side view of the flexible container with a flat bottom portion, the container comprising the aqueous oxidizing composition A.

FIG. 2: depicts a side view of the flexible container with fastening means at the bottom portion, the container comprising the aqueous oxidizing composition A.

### DETAILED DESCRIPTION OF THE INVENTION

Inventors of the present invention have unexpectedly found out that xanthines maintain the stability of flexible containers filled with aqueous oxidizing compositions A comprising hydrogen peroxide at room and elevated temperature alike. Xanthines thereby prevented premature breakage and leaking of the flexible container, maintained flexibility of the plastic material, and prevented the formation of discolorations of the plastic materials.

### Flexible Container

The present invention is directed to a cosmetic product comprising a flexible container (10, 20) and an aqueous oxidizing composition A having a pH in the range of 1 to 6 contained in the flexible container, the composition comprising:
a) one or more oxidizing agent(s), preferably hydrogen peroxide,
b) one or more compound(s) according to the following structure:

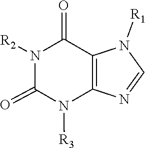

wherein R₁, R₂, and R₃ are independently selected from H and CH₃, and/or their mixtures.

It is preferred from the viewpoint of flexibility that the flexible container (10, 20) is made of thin film or deformable film as classified by ASTM D 6988. This standard method may also be used for determining the film or wall thickness of the flexible container (10, 20).

It is preferred from the viewpoint of plastic waste reduction that the flexible container (10, 20) is a plastic pouch. A plastic pouch as a special form of the flexible container (10, 20) has the lowest plastic consumption by reducing its wall thickness. Therefore, the plastic pouch is at higher risk of leakage and breakage by plastic material erosion. Premature leakage and breakage are prevented by the presently claimed oxidizing composition, in particular by adding compounds according to group b) to the oxidizing composition.

It is further preferred from the viewpoint of handling that flexible container (10, 20) has a bottom portion (11), a body (12), and a top portion, and preferably the body (12) is made of one or more polymeric material(s), the one or more polymeric material(s) preferably comprising low-density polyethylene (LDPE), high-density polyethylene (HDPE), polyethylene terephthalate (PET), and/or their recycled or biosourced material counterparts, and/or their mixtures, more preferably the one or more polymeric material(s) comprise mixtures of low-density polyethylene (LDPE) and high-density polyethylene (HDPE), polyethylene terephthalate (PET), and/or recycled or biosourced polyethylenterephthalat (PET).

It is further preferred from the viewpoint of handling that the liquid-filled, flexible container (10) is self-standing upon a bottom portion (11), preferably the bottom portion (11) is formed as a flat surface.

It is preferred from the viewpoint of container stability and container production that the flexible container (10, 20) is made of one or more polymeric material(s).

In one aspect of the present invention, it is preferred the flexible container (10, 20) is made of one or more polymeric material(s) comprising low-density polyethylene (LDPE), high-density polyethylene (HDPE), polyethylene terephthalat (PET), and/or their recycled or biosourced material counterparts, and/or their mixtures.

Particularly preferred are one or more polymeric material(s) comprising mixtures of low-density polyethylene (LDPE) and high-density polyethylene (HDPE), polyethylene terephthalat (PET), and/or recycled or biosourced polyethylenterephthalat (PET).

For this aspect it is preferred that the body (12) of the flexible container (10, 20) has a mono-layer wall comprising one or more polymeric material(s) as defined above.

For another aspect of the present invention, in particular if the flexible container (10, 20) is a pouch, it is preferred that the body (12) of the flexible container (10, 20) comprises a first inner layer of one or more polymeric material(s), a second outer layer of one or more polymeric material(s), and a barrier layer between the first inner and second outer layer of polymeric materials, with the provision that the one or more first inner and second outer layers of polymeric materials may be the same or different material, and preferably the one or more polymeric materials of the first inner and second outer layers comprise low-density polyethylene (LDPE), high-density polyethylene (HDPE), polyethylene terephthalate (PET), and/or their recycled or biosourced material counterparts, and/or their mixtures, more preferably the one or more polymeric material(s) comprise mixtures of low-density polyethylene (LDPE) and high-density polyethylene (HDPE), polyethylene terephthalate (PET), and/or recycled or biosourced polyethylenterephthalat (PET).

Premature breakage and leakage of the first inner layer is prevented by the oxidizing composition of the present invention, in particular by the addition of compound(s) according to group b). Therefore, the risk of having plastic material of the inner layer in the composition is reduced, thereby enhancing the user satisfaction, and reducing potential product complaints.

It is further preferred that the barrier layer of the flexible container (10, 20) is a SiOx-layer or an AlOx-layer. These layers may be applied to reduce water evaporation during storage and enhance the delamination of the first and second layers during recycling of the packaging material.

It is preferred from the viewpoint of container stability that the average wall thickness of the flexible container (10, 20) is 30 μm or more, more preferably 50 μm or more, still more preferably 75 μm or more.

It is preferred from the viewpoint of container flexibility that the average wall thickness of the flexible container (10, 20) is 1500 μm or less, more preferably 800 μm or less, still more preferably 500 μm or less, still further more preferably 200 μm or less.

For attaining the above-mentioned effects, it is preferred that the average wall thickness of the flexible container (10, 20) is in the range of 30 μm to 1500 μm, more preferably in the range of 30 μm to 800 μm, still more preferably in the range of 50 μm to 500 μm, still further more preferably in the range of 75 μm to 250 μm.

For measuring the average wall thickness of the container, all suitable means may be used such as optical methods based on absorption of light, ultrasonic methods, microscopic measurements, magnetic measurements, preferably light microscopic and magnetic measurements.

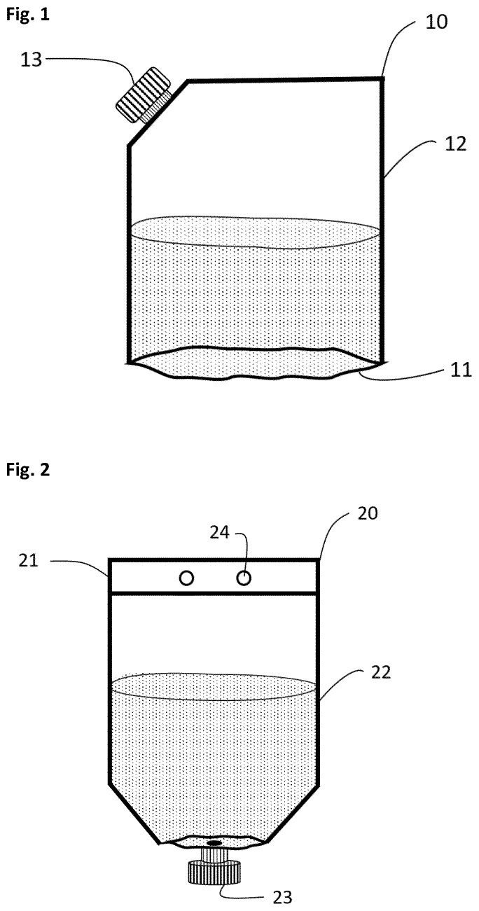

A side view of a self-standing flexible container is depicted in FIG. 1. A side view of a flexible container having fastening means is depicted in FIG. 2.

It is preferred from the viewpoint of container production that the liquid-filled, flexible container (10, 20) comprises a top portion (12, 22) extending from the bottom portion (11, 21), and one or more dispensing means (13, 23) positioned at the top portion (12, 22) of the flexible container (10, 20).

The top portion (12, 22) of the flexible container (10, 20) is made of polymeric film that is glued, fused, or heat-sealed at its top end with respect to its standing position in an upright manner to close the container in an airtight and liquid-tight fashion.

The dispensing means (13, 23) preferably is a valve allowing for dosing of the aqueous oxidizing composition A contained in the flexible container. For example, the dispensing means (13, 23) suitably is a valve, which may be optionally closed with a detachable cap. The detachable cap may be screwed or plugged onto the dispensing means (13, 23).

It is preferred from the viewpoint of product safety that the dispensing means (13, 23) has an inner end and an outer end with respect to the outside of the flexible container (10, 20), the dispensing means (13, 23) being fixed by the wall(s) of the flexible container (10, 20) such that the inner end of the dispensing means lies within the flexible container and contacts the aqueous oxidizing composition A when the container is tilted or turned upside down, and the outer end of the dispensing means extends outside of the flexible container (10, 20).

In one aspect of the present invention the liquid-filled, flexible container (10) is self-standing upon a bottom portion (11), preferably the bottom portion (11) is formed as a flat surface. This aspect is particularly advantageous for easy storage of the flexible containers (10).

In another aspect of the present invention, the liquid-filled, flexible container (20) comprises fastening means (24).

From the viewpoint of easy liquid dispensing the fastening means (24) are preferably located at the bottom portion (22) of the liquid-filled, flexible container (20), configured for fastening the container (20) in a bottom-up fashion.

Particularly suitable are fastening means (24) such as one or more latch(es), one or more flap(s), one or more through-hole(s), or one or more hook(s). The advantages of these fastening means (24) are that the liquid-filled, flexible container may be permanently positioned in a bottom-up fashion with the dispensing means (23) located toward the groups. This allows easy dispensing of the liquid, provided that the dispensing means (23) comprises an actuator configured to dispense liquid on user request and to safely seal the dispensing means in between user requests.

It is preferred from the viewpoint of environmental aspects that the flexible container is a reusable container. The term ‘reusable’ within the meaning of the present invention denotes a container that, once empty, may be refilled with the aqueous oxidizing composition A.

### Aqueous Oxidizing Composition A

The flexible container of the present invention comprises an aqueous oxidizing composition A having a pH in the range of 1 to 6, the composition comprising:
a) one or more oxidizing agent(s), preferably hydrogen peroxide,
b) one or more compound(s) according to the following structure:

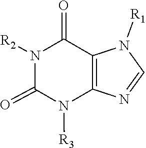

wherein R₁, R₂, and R₃ are independently selected from H and CH₃, and/or their mixtures.

It is preferred from the viewpoint of storage stability and safety of the composition that the pH of the composition is 1.25 or more, more preferably 1.5 or more, further more preferably 2 or more.

It is preferred from the viewpoint of storage stability of the composition that the pH of the composition is 5 or less, more preferably 4 or less, further more preferably 3 or less.

For attaining the above-mentioned effects, it is preferred that the pH of the composition is in the range of 1.25 to 5, more preferably in the range of 1.5 to 4, further more preferably in the range of 2 to 3.

It is further preferred from the viewpoint of product performance that the concentration of the compound according to a) is in the range of more than 1% by weight or more, more preferably 3% by weight or more, further more preferably 6% by weight or more, still more preferably 9% by weight or more, calculated to the total weight of the composition.

It is further preferred from the viewpoint of product performance and user safety that the concentration of the compound according to a) is 20% by weight or less, more preferably 15% by weight or less, further more preferably 12% by weight or less, calculated to the total weight of the composition.

For attaining the above-mentioned effects, it is preferred that the concentration of the compound according to a) is in the range of more than 1% to 20% by weight, more preferably 3% to 15% by weight, further more preferably 6% to 12% by weight, still more preferably in the range of 9% to 12% by weight, calculated to the total weight of the composition.

Suitable xanthine and/or xanthine derivatives according to the structure of compound b) are
Xanthine with R₁=R₂=R₃=H,
Theobromine with R₁=R₃=CH₃ and R₂=H,
Theophylline with R₂=R₃=CH₃ and R₁=H, and
Caffeine with R₁=R₂=R₃=CH₃.

Mixtures of the above are suitable as well.

It is preferred from economic viewpoint that at least one compound according to b) is caffeine.

It is further preferred from the viewpoint of stabilizing performance that the total concentration of compounds according to b) is 0.001% by weight or more, more preferably 0.01% by weight or more, further more preferably 0.02% by weight or more, still more preferably 0.03% by weight or more, calculated to the total weight of the composition.

It is further preferred from the viewpoint of economic reasons as well as stabilizing performance that the total concentration of compounds according to b) is 0.5% by weight or less, more preferably 0.25% by weight or less, further more preferably 0.1% by weight or less, still more preferably 0.08% by weight or less, calculated to the total weight of the composition.

For attaining the above-mentioned effects it is preferred that the total concentration of compounds according to b) is in the range of 0.001% to 0.5% by weight, preferably in the range of 0.01% to 0.25% by weight, more preferably in the range of 0.02% to 0.1% by weight, still more preferably in the range of 0.03% to 0.08% by weight, calculated to the total weight of the composition.

It is preferred from the viewpoint of stabilization performance that the weight ratio of compounds a) to b) is 2 or more, more preferably 20 or more, further more preferably 37.5 or more, further more preferably 100 or more.

It is preferred from the viewpoint of stabilization performance that the weight ratio of compounds a) to b) is 1,000 or less, more preferably 600 or less, further more preferably 400 or less, further more preferably 150 or less.

For achieving the above-mentioned effects, it is preferred that the weight ratio of compounds a) to b) is in the range of 2 to 1,000, more preferably in the range of 20 to 600, further more preferably in the range of 37.5 to 400, further more preferably in the range of 100 to 150.

### Form of Aqueous Oxidizing Composition A

The composition of the present invention preferably is an emulsion, thickened gel, or a combination thereof, from the viewpoint of cosmetic safety as well as user friendliness.

### Lipophilic Compounds as Compounds According to c)

In case the composition of the present invention is formulated as an emulsion and/or thickened emulsion, it is preferred that the composition comprises one or more lipophilic compound(s) as compound(s) according to c).

Preferably, compounds according to c) are selected from C₁₂ to C₂₂ fatty alcohols, esters of C₃ to C₂₂ alcohols with C₁₂ to C₂₂ fatty acids, C₈ to C₂₂ fatty acids, vegetable oils, and/or silicones, and/or hydrocarbon-based products, and/or their mixtures, from the viewpoint of cosmetic compatibility.

Suitable C₁₂ to C₂₂ fatty alcohols are myristyl alcohol, cetyl alcohol, stearyl alcohol, behenyl alcohol, and cetearyl alcohol.

Suitable esters of C₃ to C₂₂ alcohols with C₁₂ to C₂₂ fatty acids are isopropyl myristate, isopropyl palmitate, and myristyl myristate.

Suitable C₈ to C₂₂ fatty acids are oleic acid, linoleic acid, and palmitic acid.

Suitable vegetable oils are olive oil, almond oil, sunflower oil, and argan oil.

Suitable silicones are non-aminated and/or aminated silicones. The latter are commonly known as amodimethicones.

It is preferred from the viewpoint of forming a stable composition and user friendliness that the total concentration of compounds according to c) is 1% by weight or more, more preferably 2% by weight or more, further more preferably 3% by weight or more, calculated to the total weight of the composition.

It is preferred from the viewpoint of forming a stable composition that the total concentration of compounds according to c) is 20% by weight or less, more preferably 15% by weight or less, further more preferably 12% by weight or less, calculated to the total weight of the composition.

For attaining the above-mentioned effects, the total concentration of compounds according to c) is in the range of 1% to 20% by weight, preferably in the range of 2% to 15% by weight, more preferably in the range of 3% to 12% by weight, calculated to the total weight of the composition.

### Surfactants as Compounds According to d)

The composition of the present invention may further comprise one or more surfactant(s) as compound according to d), preferably selected from non-ionic surfactants, anionic surfactants, cationic surfactants, and/or amphoteric/zwitterionic surfactants, and/or their mixtures, more preferably selected from anionic surfactants, from the viewpoint of stabilizing the composition and improving wettability and mixability.

Preferably, the anionic surfactants may be selected from ethoxylated or non-ethoxylated alkyl ether sulfate surfactants, alkyl sulfates, ethoxylated and/or non-ethoxylated alkyl carboxylates, ethoxylated or non-ethoxylated amino acid surfactants, and/or their mixtures, and/or their salts.

Suitable examples are alkyl sulfate or preferably ethoxylated alkyl ether sulfate surfactants or mixtures thereof, and/or salts thereof, having an alkyl chain length of C₁₀ to C₂₂ and an ethoxylation degree from 1 to 50.

Suitable non-ionic surfactants may be selected from alkyl polyglycosides, ethoxylated triglycerides, ethoxylated fatty alcohols, ethoxylated fatty acid esters, ethoxylated alkyl ethers, and/or their mixtures.

Suitable cationic surfactants are quaternary ammonium surfactants having a carbon chain length in the range of C₁₂ to C₂₂ or surfactants having a tertiary amine group and at least one alkyl chain having a carbon chain length in the range of C₁₂ to C₂₂ such as alkylamidoalkylamine surfactants, and/or their salts. Suitable examples are cetrimonium chloride and behentrimonium chloride.

Suitable amphoteric/zwitterionic surfactants are of betaine type. Suitable compounds may be selected from alkyl betaines and/or alkylamido betaines. A preferred compound selected from alkyl betaines is lauryl betaine. A preferred compound selected from alkylamido betaines is cocamidopropyl betaine. The disclosure also relates to the salts of the compounds.

Suitable concentration ranges for surfactants are in the range of 0.1% to 10% by weight, calculated to the total weight of the composition, from the viewpoint of enhancing wettability of keratin fibers, physical stability, and mixability with other compositions.

### Thickening Polymers

From the viewpoint of cosmetic safety, it is further preferred that the composition of the present invention comprises one or more thickening polymer.

The composition of the present invention comprises one or more thickening polymer(s) selected from non-ionic thickening polymers and/or anionic thickening polymers, and/or their mixtures.

Preferably, the thickening polymers are selected from polymers resulting in an aqueous solution and/or aqueous dispersion at pH between 1 and 6 having a viscosity of at least 1,000 mPa·s measured at a polymer concentration of 1% by weight in water at 25° C., calculated to the total weight of the composition, determined by a Brookfield viscometer, such as at 10 rpm for 1 min, with an appropriate spindle at 25° C.

Suitable non-ionic thickening polymers are cellulose-based polymers. Suitable examples of cellulose-based polymers are methylcellulose, hydroxymethylcellulose, hydroxyethylcellulose, hydroxypropylcellulose, hydroxyethyl-methylcellulose, and alkylated hydroxyl celluloses such as (C₂-C₈)-alkylcelluloses or cetyl hydroxyethylcellulose.

Suitable anionic thickening polymers are selected from naturally-based anionic polymers and/or synthetic anionic polymers.

Suitably, the natural anionic polymer(s) may be selected from xanthan gum, dehydroxanthan gum, hydroxypropylxanthan gum, carboxymethyl cellulose and starch based polymers such as vegetable starch and/or their synthetically modified derivatives such as hydroxypropyl starch phosphate. Equally suitable are alginic acids, sodium alginates, ammonium alginates, calcium alginates, gum arabic, and guar gum.

Suitable synthetic anionic polymers are associative thickening polymers, such as acrylates/steareth-30 methacrylate copolymer.

The preferred thickening polymer for the composition of the present invention are natural anionic polymers, more preferably xanthan gum and/or dehydroxanthan gum, from the viewpoint of their biodegradability and low environmental impact.

Preferably, the total concentration of thickening polymers of the present invention are 0.1% by weight or more, more preferably 0.25% by weight or more, more preferably 0.5% by weight or more, calculated to the total weight of the composition, from the viewpoint of providing sufficient viscosity to the composition.

Preferably, the total concentration of thickening polymers of the present invention are 15% by weight or less, more preferably 12% by weight or more, further more preferably 10% by weight or less, calculated to the total weight of the composition, from the viewpoint of providing sufficient viscosity to the composition and cost of goods.

For attaining the above-mentioned effects, it is preferred that the total concentration of thickening polymers in the composition of the present invention is in the range of 0.1% to 15% by weight, preferably in the range of 0.25% to 12% by weight, more preferably in the range of 0.5% to 10% by weight, calculated to the total weight of the composition.

It is preferred from the viewpoint of cosmetic safety that the composition of the present invention has a viscosity in the range of 1,000 Pas to 25,000 mPas, preferably 2,000 mPas to 20,000 mPas, more preferably in the range of 2,500 mPas to 17,500 mPas, determined by cone plate viscometry at 25° C. under atmospheric conditions. A suitable viscometer is a Brookfield viscometer with spindle #4.

### Process of Making the Flexible Container

The present invention is also directed to a process of making the cosmetic product having a flexible container (10, 20) filled with an aqueous oxidizing composition A comprising the steps of:
(i) forming a closed flexible container as defined above from a film material and fixing the dispensing means (13, 23) to or between the walls of the flexible container,
(ii) filling the flexible container with the aqueous oxidizing composition A as defined above by injecting the liquid composition into the flexible container through the dispensing means (13. 23),
(iii) sealing the flexible container by closing the dispensing means (13, 23), preferably with a detachable cap.

The sealing step iii) is preferably a heat-sealing or ultrasonic sealing step.

### Kit-of-Parts

The present invention is also directed to a kit-of-parts comprising the cosmetic product as defined above and one more composition selected from:
an aqueous composition B having a pH in the range of 7 to 12 and comprising one or more oxidative dye precursors and/or oxidative dye couplers,
a bleach powder composition C comprising one or more persalt(s) and/or peroxy salt(s) and one or more alkalizing agent(s),
an aqueous lightening composition D comprising one or more alkalizing agent(s) and having a pH in the range of 7 to 12,
an aqueous composition E having a pH in the range of 3 to 12 comprising one or more reducing agent(s) and one or more alkalizing agent(s).

It is preferred from the viewpoint of customer friendliness that the aforementioned compositions are separately packed.

The following examples are to illustrate the invention, but not to limit it.

### Examples

### Table 1

The following compositions were prepared by dissolving caffeine as compound according to b) in an aqueous composition of hydrogen peroxide under constant stirring:

| | Inv. comp. 1 | Inv. | Compar. | Compar. |
|---|---|---|---|---|
| | [% by weight] | comp. 2 | Comp. 1 | Comp. 2 |
| Ingredient | [% by weight] | | | |
| Caffeine | 0.05 | | — | |
| Hydrogen peroxide | 9.0 | 4.5 | 9.0 | 4.5 |

| | |
|---|---|
| Phosphoric acid | q.s. ad pH 2.5 |
| Water | Ad 100.0 |

The compositions were prepared and stored under controlled conditions at 50° C. for 2 months. 5 g Pieces of the following plastic materials were immersed in 500 mL of the oxidative compositions from above.
1: PP/Adflex: Polypropylene (80% w/w, PP Borealis RB 307 MO) and Adflex Q 300F (20% w/w), 770 μm thickness
2: HDPE/LDPE: LyondellBasell Hostalen ACP 5231 D (75% w/w) and Dow Chemical Dowlex 2045 G (25% w/w), 960 μm thickness
3: PET: Indorama Ramapet N1(S), 570 μm thickness
4: Recycled Polyethylene Terephthalate (PET), 250 μm thickness

| | | 1: | 2: | 3: | 4: |
|---|---|---|---|---|---|
| Composition | Feature | PP/Adflex | HDPE/LDPE | PET | rPET |
| Inv. 1 | Roughness | 2 | 2 | 3 | 2 |
| | Flexibility | 3 | 2 | 3 | 3 |
| Inv. 2 | Roughness | 2 | 2 | 3 | 2 |
| | Flexibility | 3 | 2 | 3 | 3 |
| Compar. 1 | Roughness | 2 | 0 | 1* | 1* |
| | Flexibility | 2 | 2 | 0* | 0* |
| Compar. 2 | Roughness | 2 | 0 | 1* | 1* |
| | Flexibility | 2 | 2 | 0* | 0* |

Roughness: 0 (very rough) to 5 (very smooth)
Flexibility: 0 (inflexible, material breaks) to 5 (complete flexibility)
*Strong shape-change of material, yellow stains

### Methods

For the examples of table 1, the plastic materials were touched by 5 independent experts without knowing the material or treatment group. They were asked to evaluate the roughness and flexibility of the materials and to assign an integer number. Roughness was evaluated on a scale of 0 (very rough) to 5 (very smooth), flexibility was rated on a scale 0 (inflexible, material breaks) to 5 (complete flexibility).

The reported values are mode values of the expert classifications.

For the example of table 2, the evaluation was done by qualitative assessment of the stored plastic samples.

The following additional plastic materials were tested:
5: 100% LDPE, 1.2 mm thickness
6: 100% HDPE, 0.92 mm thickness

TABLE 2

| | Comp. 3 | Inv. 3 | Inv. 4 | Comp. 4 | Inv. 5 | Inv. 6 | Inv. 7 | Inv. 8 | Inv. 9 | Inv. 10 |
|---|---|---|---|---|---|---|---|---|---|---|
| Ingredients | [% by weight] | | | | | | | | | |
| Caffeine | — | 0.001 | 0.5 | — | — | — | — | — | — | — |
| Theobromine | — | — | — | — | 0.001 | 0.5 | — | — | — | 0.1 |
| Xanthine | — | — | — | — | — | — | 0.001 | 0.5 | 0.1 | — |
| Hydrogen peroxide | 12.0 | 12.0 | 12.0 | 12.0 | 12.0 | 12.0 | 12.0 | 12.0 | 12.0 | 12.0 |
| Phosphoric acid | q.s. ad pH 2.5 | | | | | | | | | |
| Water | Ad 100.0 | | | | | | | | | |
| Plastic material | [5] | [5] | [5] | [6] | [6] | [6] | [6] | [6] | [4] | [4] |
| type | | | | | | | | | | |
| | Evaluation | | | | | | | | | |
| Roughness | Rougher | No change | No change | Rubber-like | No change | No change | No change | No change | Defined | Defined |
| | feel | compared | compared | surface | compared | compared | compared | compared | shape | shape |
| | compared | to fresh | to fresh | | to fresh | to fresh | to fresh | to fresh | remains | remains |
| | to fresh | material | material | | material | material | material | material | intact** | intact** |
| | material | | | | | | | | | |
| Flexibility | Brittle, | No change | No change | Softer, | No change | No change | No change | No change | No rolling | No rolling |
| | stiffe | compared | compared | easier to | compared | compared | compared | compared | up, | up, |
| | compared | to fresh | to fresh | bend | to fresh | to fresh | to fresh | to fresh | flexibility | flexibility |
| | to fresh | material | material | compared | material | material | material | material | compared | compared |
| | material | | | to fresh | | | | | to fresh | to fresh |
| | | | | material | | | | | material | material |

**plastic material remained clear, no yellow stains

1. A cosmetic product comprising
a flexible container and
an aqueous oxidizing composition A having a pH in a range of 1 to 6 contained in the flexible container, the composition comprising:
at least one oxidizing agent, and
at least one compound according to the following Formula (I): 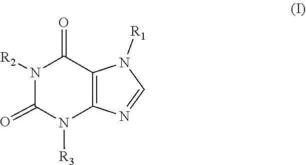
wherein R₁, R₂, and R₃ are each independently selected from H and CH₃.

2. The cosmetic product according to claim 1, wherein the flexible container is a plastic pouch.

3. The cosmetic product according to claim 1, wherein
the flexible container has a bottom portion, a body, and a top portion, and
the body is made of at least one polymeric material selected from the group consisting of low-density polyethylene (LDPE), high-density polyethylene (HDPE), polyethylene terephthalate (PET), and recycled or biosourced material counterparts thereof.

4. The cosmetic product according to claim 1, wherein the flexible container is self-standing upon a bottom portion, which is formed as a flat surface.

5. The cosmetic product according to claim 1, wherein the flexible container is made of at least one polymeric material selected from the group consisting of low-density polyethylene (LDPE), high-density polyethylene (HDPE), polyethylene terephthalate (PET), and recycled or biosourced material counterparts thereof.

6. The cosmetic product according to claim 3, wherein
the body of the flexible container comprises a first inner layer of at least one polymeric material, a second outer layer of at least one polymeric material, and a barrier layer between the first inner and second outer layer,
the first inner layer and second outer layer may be the same or different polymeric material, and
the polymeric materials of the first inner and second outer layers are each independently selected from the group consisting of low-density polyethylene (LDPE), high-density polyethylene (HDPE), polyethylene terephthalate (PET), and recycled or biosourced material counterparts thereof.

7. The cosmetic product according to claim 6, wherein the barrier layer is a SiOx-layer or an AlOx-layer.

8. The cosmetic product according to claim 1, wherein an average wall thickness of the flexible container is in a range of 50 μm to 500 μm.

9. The cosmetic product according to claim 1, wherein the flexible container comprises one or more dispensing means positioned at a top portion of the flexible container, the dispensing means having an inner end and an outer end with respect to an outside of the flexible container, the dispending means being fixed by a wall of the flexible container such that the inner end of the dispensing means lies within the flexible container and contacts the aqueous oxidizing composition A when the flexible container is tilted or turned upside down, and the outer end of the dispensing means extends outside of the flexible container.

10. The cosmetic product according to claim 1, wherein the flexible container comprises a fastening means positioned at a bottom portion of the flexible container, the fastening means being at least one selected from the group consisting of a latch, a flap, a through-hole, and a hook.

11. (canceled)

12. (canceled)

13. The cosmetic product according to claim 9, wherein the dispensing means comprises an actuator configured to open the dispensing means for dispensing the aqueous oxidizing composition A upon a user's request.

14. The cosmetic product according to claim 1, wherein a concentration of the oxidizing agent in the aqueous oxidizing composition A is in a range of 1% to 20% by weight, based on a total weight of the aqueous oxidizing composition A.

15. The cosmetic product according to claim 1, wherein the at least one compound according to Formula (I) is caffeine.

16. The cosmetic product according to claim 1, wherein a total concentration of compounds according to Formula (I) in the aqueous oxidizing composition A is in a range of 0.001% to 0.5% by weight, based on a total weight of the aqueous oxidizing composition A.

17. The cosmetic product according to claim 1, wherein the aqueous oxidizing composition A further comprises at least one lipophilic compound selected from the group consisting of C₁₂ to C₂₂ fatty alcohols, esters of C₃ to C₂₂ alcohols with C₁₂ to C₂₂ fatty acids, C₈ to C₂₂ fatty acids, vegetable oils, silicones, and hydrocarbon-based products.

18. (canceled)

19. A process of making a cosmetic product having a flexible container filled with an aqueous oxidizing composition A, the method comprising:
forming a closed flexible container from a film material and fixing a dispensing means to or between walls of the flexible container,
filling the flexible container with an aqueous oxidizing composition A by injecting the aqueous oxidizing composition A into the flexible container through the dispensing means, and
sealing the flexible container by closing the dispensing means with a detachable cap, wherein
the aqueous oxidizing composition A has a pH in a range of 1 to 6 and comprises at least one oxidizing agent and at least one compound according to the following Formula (I): 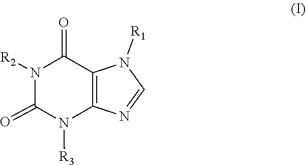
wherein R₁, R₂, and R₃ are each independently selected from H and CH₃.

20. A kit-of-parts comprising
the cosmetic product of claim 1 and
one more composition selected from the group consisting of:
an aqueous composition B having a pH in a range of 7 to 12 and comprising at least one oxidative dye precursor and/or at least one oxidative dye coupler,
a bleach powder composition C comprising at least one persalt and/or at least one peroxy salt and at least one alkalizing agent,
an aqueous lightening composition D comprising at least one alkalizing agent and having a pH in a range of 7 to 12, and
an aqueous composition E having a pH in a range of 3 to 12 and comprising at least one reducing agent and at least one alkalizing agent.

21. The cosmetic product according to claim 1, wherein the at least one oxidizing agent is hydrogen peroxide.

22. The cosmetic product according to claim 1, wherein a concentration of the at least one oxidizing agent in the aqueous oxidizing composition A is in a range of 6% to 12% by weight, based on a total weight of the aqueous oxidizing composition A.

23. The cosmetic product according to claim 1, wherein a total concentration of compounds according to Formula (I) in the aqueous oxidizing composition A is in a range of 0.03% to 0.08% by weight, based on a total weight of the aqueous oxidizing composition A.
